(19) United States
(12) Patent Application Publication
(10) Pub. No.: US 2024/0367974 A1
(43) Pub. Date: Nov. 7, 2024

(54) BORON NITRIDE CONTAINING VACANT SITE DEFECTS FOR USE IN CATALYTIC HYDROGENATION

(71) Applicant: UT-Battelle, LLC, Oak Ridge, TN (US)

(72) Inventors: Sheng Dai, Knoxville, TN (US); Zhenzhen Yang, Knoxville, TN (US); Hao Chen, Changsha (CN)

(21) Appl. No.: 18/653,397

(22) Filed: May 2, 2024

Related U.S. Application Data
(60) Provisional application No. 63/463,616, filed on May 3, 2023.

(51) Int. Cl. C01B 21/064; B01J 27/24; B01J 37/08; C07C 5/05

(52) U.S. Cl. CPC C01B 21/0646; B01J 27/24; B01J 37/088; C07C 5/05; C01P 2002/50; C01P 2002/76; C07C 2521/02; C07C 2527/24

(57) ABSTRACT

A composition comprising a boron nitride hexagonal lattice structure in which boron atoms and nitrogen atoms are present in a B:N molar ratio of 1:4-1:8 or 4:1-8:1, wherein the molar ratio corresponds to vacant site defects within the boron nitride hexagonal lattice structure. Also described are methods for producing the boron nitride composition as well as methods for using the boron nitride composition as a catalyst in a hydrogenation process.

### CROSS-REFERENCE TO RELATED APPLICATIONS

The present application claims benefit of U.S. Provisional Application No. 63/463,616, filed May 3, 2023, all of the contents of which are incorporated herein by reference.

### STATEMENT REGARDING FEDERALLY SPONSORED RESEARCH

This invention was made with government support under Prime Contract No. DE-AC05-00OR22725 awarded by the U.S. Department of Energy. The government has certain rights in the invention.

### FIELD OF THE INVENTION

The present invention relates to the fields of catalytic hydrogenation, hydrogenation catalysts (particularly metal-free catalysts), boron nitride compositions, and catalysts containing frustrated Lewis pairs (FLPs).

### BACKGROUND

Catalytic hydrogenations constitute a prime technology for the conversion of fine and bulk chemicals to their counterparts with reduced redox state. However, catalytic hydrogenations generally rely on rare elements (e.g., Pd, Pt, Rh, Ru, In or Ce). Some of these metals (e.g., In or Ce) act as a Lewis acid in adequate distance from base sites (e.g., OH or O). Such rare elements are generally costly and in limited supply. Present day hydrogenation catalysts also generally need to be operated at elevated temperatures (typically, over 200° C. or 250° C.) and elevated pressure (above 1, 5, or 10 atm). Efforts in developing a hydrogenation catalyst with lower reliance on rare elements and that can be operated at more moderate temperatures and pressures have been largely unsuccessful. Thus, developing such a hydrogenation catalyst would greatly benefit the hydrogenation process and would thus represent a significant advance in the field of catalytic hydrogenation technology.

### SUMMARY

In one aspect, the present disclosure is directed to a unique form of hexagonal boron nitride in which the ordered lattice structure is punctuated by the presence of highly reactive vacant site defects, also referred to herein as frustrated Lewis pairs (FLPs), unsaturated sites, or unbonded or Lewis B or N sites. As further discussed below, the vacant site defects arise by employing non-stoichiometric molar ratios of boron and nitrogen during the production process. More specifically, the hexagonal boron nitride contains boron atoms and nitrogen atoms present in a B:N molar ratio of 1:4-1:8 or 4:1-8:1 (or more particularly, 1:5-1:7 or 5:1-7:1, or 1:4-1:8, or 1:5:1:7), wherein the foregoing molar ratios provide vacant site defects within the boron nitride hexagonal lattice structure. The hexagonal boron nitride material described herein is advantageously free of costly metals (i.e., “metal-free”) and can be operated at more moderate temperatures and at a lower pressure, which may even be ambient pressure (about 1 atm).

In another aspect, the present disclosure is directed to a method of producing the above described hexagonal boron nitride catalyst containing lattice defects. In preferred embodiments, the method includes heating a molten mixture of NaBH₄ and NaNH₂ under an inert atmosphere to a temperature of at least 700° C., wherein the molar ratio of NaBH₄:NaNH₂ is 1:4-1:8 or 4:1-8:1. In some embodiments, the temperature is at least 750° C. or 800° C. In further or alternative embodiments, the molar ratio of NaBH₄:NaNH₂ may be, for example, 1:5:1:7 or 5:1-7:1, or the molar ratio may be 1:4-1:8, or the molar ration may be 1:5:1:7.

In another aspect, the present disclosure is directed to a method of catalytically hydrogenating an unsaturated organic compound by contacting the above described boron nitride catalyst with hydrogen gas in the presence of a hydrogenation catalyst at a temperature of 80° C.-300° C. In some embodiments, the temperature is more specifically in a range of 80° C.-200° C. or 80° C.-150° C. The catalyst may also have any of the B:N molar ratios provided anywhere above. In typical embodiments, for any of the temperature ranges given, the catalyst may be operated at reduced pressure (e.g., up or less than 5 atm) or at ambient pressure.

### BRIEF DESCRIPTION OF THE DRAWINGS

FIG. 1 is a schematic of an ionothermal process for producing an h-BN material containing vacant site defects. The schematic also shows the nature of the defects and their involvement in H₂ activation and dissociation.

FIGS. 2A-2G. FIG. 2A shows B K-edge NEXAFS spectra while FIG. 2B shows N K-edge NEXAFS spectra of the h-BNs produced and studied in this work. The inset in FIG. 2A shows the existence of boron sites possessing unsaturated bonding types. FIG. 2C shows PXRD patterns of the h-BNs produced and studied in this work. FIG. 2D shows pair distribution functions (PDFs) of the h-BNs produced and studied in this work. The insets (left and right) in FIG. 2D show the peak at r=2.0 Å for h-BN-4, being assigned to the specific B-B distances from the medium size BN2 vacancies, while this feature was absent or much less pronounced for h-BN-1 and h-BN-5, respectively. FIG. 2E shows FTIR spectra of the h-BNs produced and studied in this work. FIG. 2F shows solid state 11B NMR of the h-BNs produced and studied in this work. FIG. 2G is a transmission electron microscope (TEM) image of h-BN-4. The h-BN materials were produced by thermal treatment of NaBH₄ and NaNH₂ mixtures with various NaBH₄:NaNH₂ molar ratios including 1:1, 1:3, 1:5, 1:7, and 1:9, and all these batches afforded high-quality h-BN materials denoted as h-BN-1, h-BN-2, h-BN-3, h-BN-4, and h-BN-5, respectively.

FIGS. 3A-3I. FIGS. 3A and 3B show NH₃-TPD and CO₂-TPD profiles, respectively, of the following series of h-BNs produced and studied in this work: h-BN-1, h-BN-2, h-BN-3, h-BN-4, and h-BN-5. FIG. 3C shows H/D exchange detected by ¹H NMR (d₁₂-cyclohexane, 400 MHZ) at 100° C. and 1 bar H₂/D₂ (1:1) catalyzed by h-BN-1. FIG. 3D shows H/D exchange detected by ¹H NMR (d₁₂-cyclohexane, 400 MHZ) at 100° C. and 1 bar H₂/D₂ (1:1) catalyzed by h-BN-4. The numbers shown in parentheses correspond to the molar ratio of HD:H₂. FIG. 3E shows H/D exchange detected by ¹H NMR (d₁₂-cyclohexane, 400 MHZ) at 100° C. and 1 bar H₂/D₂ (1:1) catalyzed by h-BN-5. FIGS. 3F, 3G, and 3H show in situ DRIFTS spectra of H₂ chemisorption on h-BN-1, h-BN-4, and h-BN-5, respectively. FIG. 3I is a graph showing mass fragments of the outlet gas stream derived from H₂/D₂ chemisorption on h-BN-4.

FIGS. 4A-4B. FIG. 4A provides a schematic and data showing catalytic activity of h-BNs in styrene hydrogenation. FIG. 4B is a schematic showing isotope-labeling experiments catalyzed by h-BN-4 under H₂/D₂ atmosphere.

### DETAILED DESCRIPTION

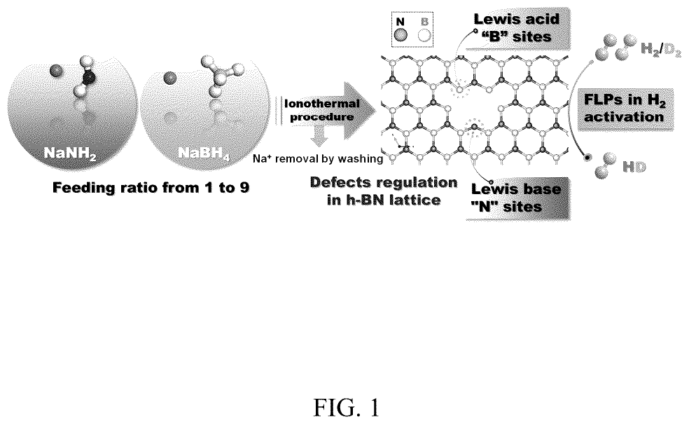

As well known, conventional two-dimensional (2D) hexagonal boron nitride (h-BN) is prepared using equimolar amounts of B and N atoms and possesses alternating surface Lewis acid site (B center) and Lewis base site (N center) with a layered honeycomb-like structure and a substantial absence of defects. In contrast, the presently described h-BN material, which is prepared using non-equimolar amounts of B and N, is characterized by a substantial presence of vacant site defects (i.e., FLP-like sites), such as depicted in FIG. 1. The vacant site defects can be understood as breaks or disruptions of the h-BN lattice structure resulting from a lack of N atoms (i.e., less than three) bonding to B or a lack of B atoms (i.e., less than three) bonding to N. B atoms bonded to less than three N atoms can be considered Lewis acid (“B”) sites while N atoms bonded to less than three B atoms can be considered Lewis base (“N”) sites. Notably, it has herein been surprisingly found that, although conventional h-BN has little to no hydrogenation ability, the presently described h-BN material possesses substantial hydrogenation ability, even under relatively milder temperature and/or pressure conditions than the conventional h-BN material.

In a first aspect, the present disclosure is directed to a catalyst composition containing a boron nitride hexagonal (h-BN) lattice structure in which boron atoms and nitrogen atoms are present in a B:N molar ratio of 1:4-1:8 or 4:1-8:1 and wherein the boron nitride hexagonal lattice structure contains vacant site defects. Since the foregoing molar ratios do not include an equimolar (1:1) ratio, they result in or give rise to (i.e., correspond to) vacant site defects within the boron nitride hexagonal lattice structure.

In a first set of embodiments, the B:N molar ratio is within a range of 1:4-1:8. The B:N molar ratio may more particularly be precisely or about, for example, 1:4, 1:5, 1:6, 1:7, or 1:8, or a range bounded by any two of these values, e.g., 1:4-1:8, 1:4-1:7, 1:4-1:6, 1:4-1:5, 1:5-1:8, 1:5-1:7, 1:5-1:6, 1:6-1:8, 1:6-1:7, or 1:7-1:8.

In a second set of embodiments, the B:N molar ratio is within a range of 4:1 to 8:1. The B:N molar ratio may more particularly be precisely or about, for example, 4:1, 5:1, 6:1, 7:1, or 8:1, or a range bounded by any two of these values, e.g., 4:1-8:1, 4:1-7:1, 4:1-6:1, 4:1-5:1, 5:1-8:1, 5:1-7:1, 5:1-6:1, 6:1-8:1, 6:1-7:1, or 7:1-8:1.

In some embodiments, the molar ratio is exclusively within any of the above ranges of molar ratios, and thus, excludes any molar ratios outside of the range. In some embodiments, any one or more molar ratios or ranges in molar ratios, such as provided above, are excluded. Moreover, the B:N molar ratio may, in some embodiments, be within a range bounded by any molar ratio found in the above first set of embodiments and any molar ratio found in the above second set of embodiments, except that a molar ratio of 1:1 or perhaps a molar ratio in a range of 1:2-2:1 is excluded. For example, the B:N molar ratio may be within a range of 1:4-4:1 or 1:6-6:1, excluding a molar ratio of 1:1 or excluding a molar ratio in a range of 1:2-2:1.

Typically, the h-BN material contains nanopores, i.e., is nanoporous. The nanopores typically have a size of 0.5-5 nm, or more particularly, a size of 0.5-3 nm or 0.5-2 nm. The foregoing sizes may correspond to lattice interatom distances or the size of defects. The size of vacant site defects may, in some cases, be larger than other nanopores in the material, e.g., 3, 4, 5, 6, 7, or 10 nm or within a range bounded by any two of the foregoing values. The nanopores may have any of a variety of shapes, such as a circular, square, or triangular shape.

In another aspect, the present disclosure is directed to methods for producing the presently described h-BN material containing vacant site defects as described above. In the method, a molten mixture of NaBH₄ and NaNH₂ is heated under an inert atmosphere to a temperature of at least 700° C., wherein the molar ratio of NaBH₄:NaNH₂ is 1:4-1:8 or 4:1-8:1 or any of the other molar ratios or ranges thereof provided earlier above, excluding a molar ratio of 1:1 or perhaps a ratio in a range of 1:2-2:1. The inert atmosphere is typically substantially devoid of oxygen or any other reactive gas and is typically predominantly or exclusively composed of one or more inert gases, such as nitrogen and/or argon. In various embodiments, the final temperature at which the molten mixture is heated is precisely or about, for example, 700° C., 725° C., 750° C., 775° C., 800° C., 825° C., 850° C., 875° C., or 900° C., or the temperature is within a range bounded by any two of the foregoing values. Typically, the molten mixture is heated at any of the foregoing final temperatures for a period of time, which is typically precisely or at least 0.5 hour, or more typically, precisely or at least 1 hour, 1.5 hours, 2 hours, 2.5 hours, or 3 hours. In some embodiments, the temperature is raised from room temperature to an intermediate temperature (e.g., 400-600° C., 450-550° C., or about 500° C.) at which the molten mixture is maintained for precisely or at least 0.5, 1, or 1.5 hours, followed by raising the temperature, typically at a ramp rate (e.g., precisely, at least, or up to 1, 2, 5, or 10° C./min) to the final temperature of precisely or about, for example, 700° C., 725° C., 750° C., 775° C., 800° C., 825° C., 850° C., 875° C., or 900° C. Typically, the resulting solid material is washed with deionized water to remove sodium-containing byproduct e.g., NaH. Following the washing step, the solid material is typically dried (typically at a temperature of at least 50° C. and up to 150° C.) for at least 3, 6, 9, or 12 hours.

In a first set of embodiments, the NaBH₄:NaNH₂ molar ratio is within a range of 1:4-1:8. The NaBH₄:NaNH₂ molar ratio may more particularly be precisely or about, for example, 1:4, 1:5, 1:6, 1:7, or 1:8, or a range bounded by any two of these values, e.g., 1:4-1:8, 1:4-1:7, 1:4-1:6, 1:4-1:5, 1:5-1:8, 1:5-1:7, 1:5-1:6, 1:6-1:8, 1:6-1:7, or 1:7-1:8.

In a second set of embodiments, the NaBH₄:NaNH₂ molar ratio is within a range of 4:1 to 8:1. The NaBH₄:NaNH₂ molar ratio may more particularly be precisely or about, for example, 4:1, 5:1, 6:1, 7:1, or 8:1, or a range bounded by any two of these values, e.g., 4:1-8:1, 4:1-7:1, 4:1-6:1, 4:1-5:1, 5:1-8:1, 5:1-7:1, 5:1-6:1, 6:1-8:1, 6:1-7:1, or 7:1-8:1.

In some embodiments, the NaBH₄:NaNH₂ molar ratio is exclusively within any of the above ranges of molar ratios, and thus, excludes any molar ratios outside of the range. In some embodiments, any one or more molar ratios or ranges in molar ratios, such as provided above, are excluded. Moreover, the NaBH₄:NaNH₂ molar ratio may, in some embodiments, be within a range bounded by any molar ratio found in the above first set of embodiments and any molar ratio found in the above second set of embodiments, except that a molar ratio of 1:1 or perhaps a molar ratio in a range of 1:2-2:1 is excluded. For example, the NaBH₄:NaNH₂ molar ratio may be within a range of 1:4-4:1 or 1:6-6:1, excluding a molar ratio of 1:1 or excluding a molar ratio in a range of 1:2-2:1.

In another aspect, the present disclosure is directed to a method of catalytically hydrogenating an unsaturated organic compound by use of the above described h-BN catalyst containing a substantial amount of defects. The unsaturated organic compound may be, for example, an olefin, alkyne, unsaturated oil, aromatic, ketone, aldehyde, imine, nitrile, ester, or carboxylic acid. The reaction temperature is typically at least 80° C. and up to 300° C. In various embodiments, the reaction temperature may be, for example, precisely or about 80° C., 90° C., 100° C., 110° C., 120° C., 130° C., 140° C., 150° C., 180° C., 200° C., 220° C., 250° C., 280° C., or 300° C., or the temperature is within a range bounded by any two of the foregoing values (e.g., 80° C.-200° C. or 80° C.-150° C.). The h-BN catalyst may have any of the B:N molar ratios described above, such as 1:4-1:8, 4:1-8:1, 1:5:1:7, or 5:1-7:1 or any of the other specific values or sub-ranges provided earlier above.

Examples have been set forth below for the purpose of illustration and to describe certain specific embodiments of the invention. However, the scope of this invention is not to be in any way limited by the examples set forth herein.

### Examples

### Overview

In this work, H₂ activation and dissociation catalyzed by h-BN was achieved by incorporating into the h-BN lattice sterically-hindered vacant defect Lewis acid (“B” center) and Lewis base (“N” center) sites anchored within the rigid lattice of the h-BN scaffold. The concept is schematically shown in FIG. 1. Using the molten salts-derived ionothermal method developed herein, the concentration of vacant defect sites can advantageously be controlled by judicious selection of the feeding ratio of nitrogen (NaNH₂) and boron (NaBH₄) source. The highly crystalline and nanoporous h-BN materials developed herein were capable of splitting H₂/D₂ under ambient pressure via FLP-like behavior. In some embodiments, the h-BN materials developed herein also exhibited a catalytic efficiency surpassing state-of-the-art heterogeneous metal-free analogues.

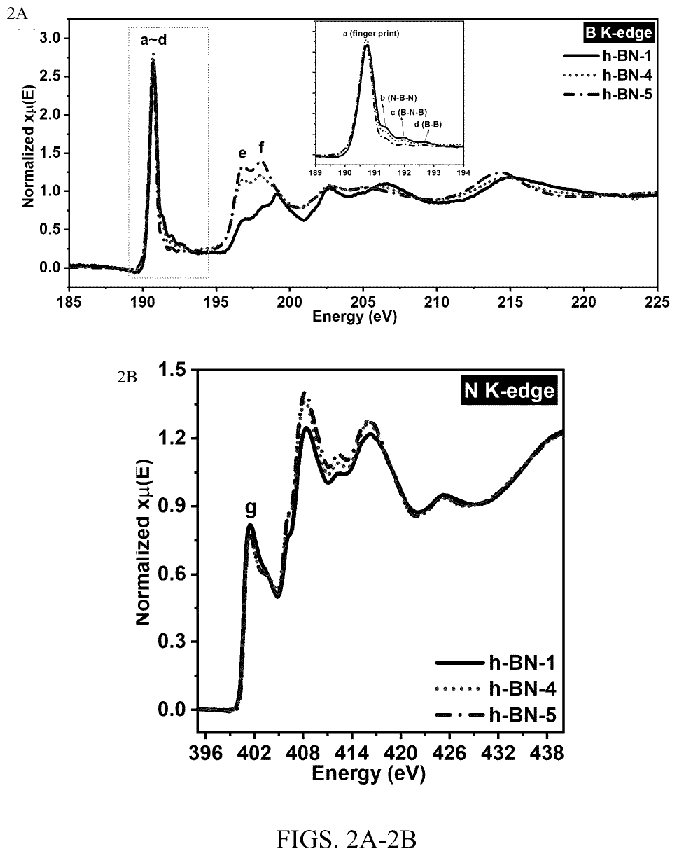

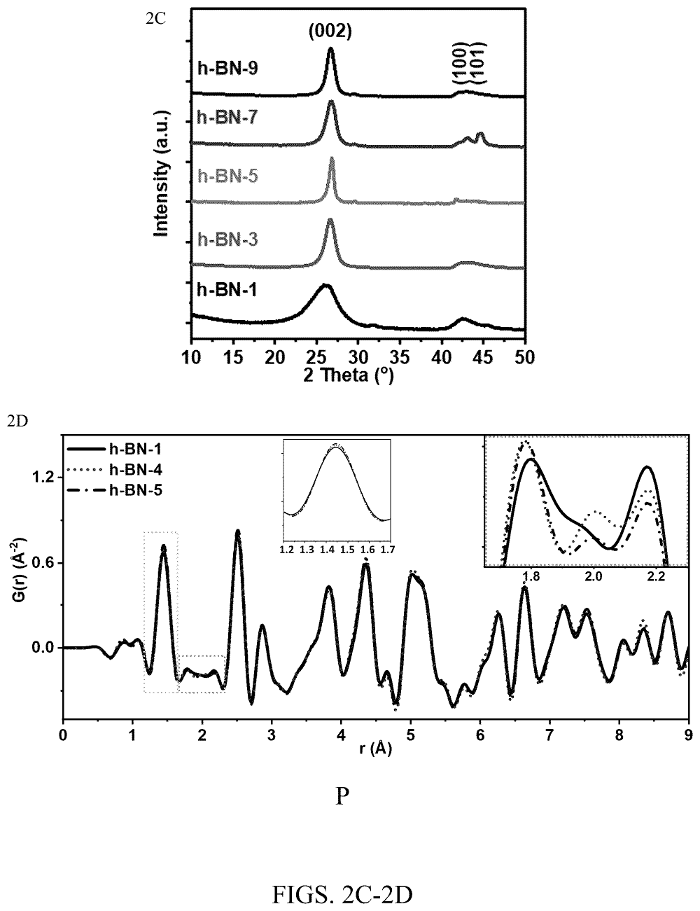

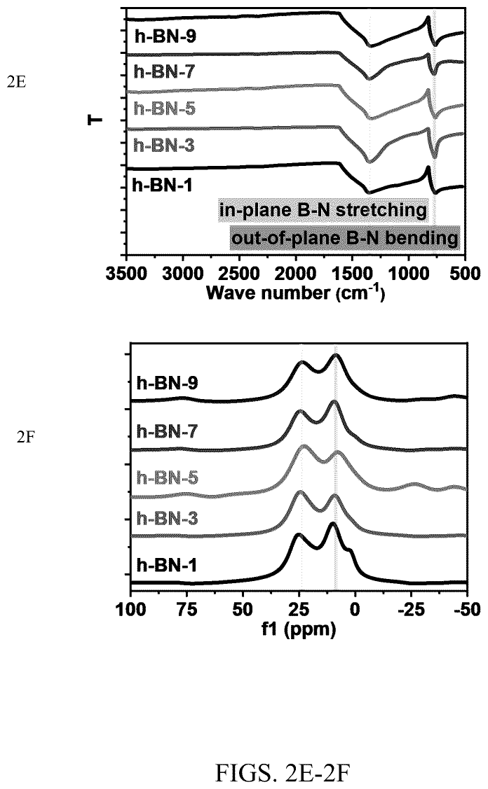

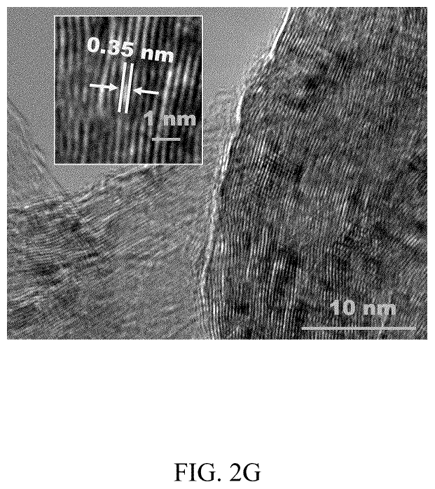

In the molten salts-involved ionothermal procedure, equimolar NaBH₄ and NaNH₂ reactants were employed as boron and nitrogen sources, respectively. Theoretically, varying the ratio of the starting materials, e.g., by adding more boron source (NaBH₄) or nitrogen source (NaNH₂), will generate h-BNs with diverse defects within the lattice, thus creating fixed Lewis acid (B) and base (N) sites with an unsaturated bonding type. To demonstrate this, thermal treatment of the NaBH₄ and NaNH₂ mixtures with various molar ratios, including 1:1, 1:3, 1:5, 1:7, and 1:9, was conducted, and all these batches afforded high-quality h-BN materials denoted as h-BN-1, h-BN-2, h-BN-3, h-BN-4, and h-BN-5, respectively. Near-edge X-ray absorption fine structure (NEXAFS) spectra of the as-afforded h-BNs demonstrated the successful introduction of B and N defects within the h-BN scaffolds with controllable types and ratios. Results for h-BN-1, h-BN-4, and h-BN-5 are shown in FIGS. 2A and 2B.

Results of powder X-ray diffraction (PXRD) verified that the atomically periodic architecture of h-BNs matched well with a typical hexagonal structure (JCPDS card no. 01-073-2095) (H. Chen et al., Angew. Chem. Int. Ed., 58 (31), 10626, 2019). The PXRD results for h-BN-1, h-BN-2, h-BN-3, h-BN-4, and h-BN-5 are shown FIG. 2C. A strong diffraction (002) peak at 20=26.31° corresponds to the interplanar distance of 3.5 Å. Other relatively weak diffraction peaks at 20=42.43° and 45.21° can be assigned to the (100) and (101) crystal planes, respectively, which indicates an extended/ordered stacking in the c-direction. As shown in FIG. 2D, pair distribution function (PDF) analysis clearly showed the peak at r=2.0 Å for h-BN-4, being assigned to the specific B-B distances from the medium size BN2 vacancies, while this feature was absent or much less pronounced for h-BN-1 and h-BN-5, respectively.

The chemical structures of the as-afforded h-BNs were characterized by Fourier transform infrared (FTIR) spectra, as shown in FIG. 2E, and magic angle spinning ¹¹B solid-state nuclear magnetic resonance (NMR) spectra, as shown in FIG. 2F. Microscopy images of h-BN-4, as shown in FIG. 2G, reveal that it is composed of crystalline sheet-like flakes with a thickness of ˜10 nm and 20˜25 atomic layers in each sheet. The five h-BNs also exhibited similar Brunauer-Emmett-Teller (BET) surface areas in the range of 55˜71 m² g⁻¹, as detected by N₂ isotherms at 77 K. Inductively coupled plasma atomic emission analysis (ICP-AES) revealed that no detectable Ni (<0.0009 ppm) was observed and the residual Na⁺ content was below 0.1 wt % for all h-BNs.

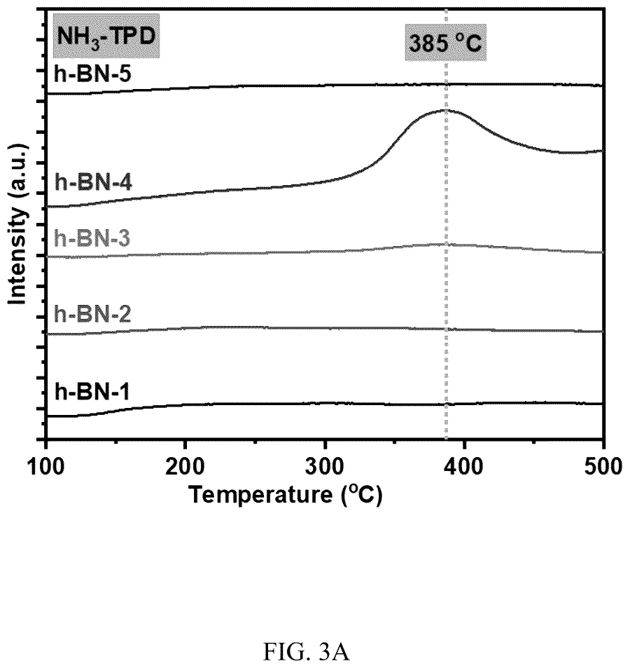

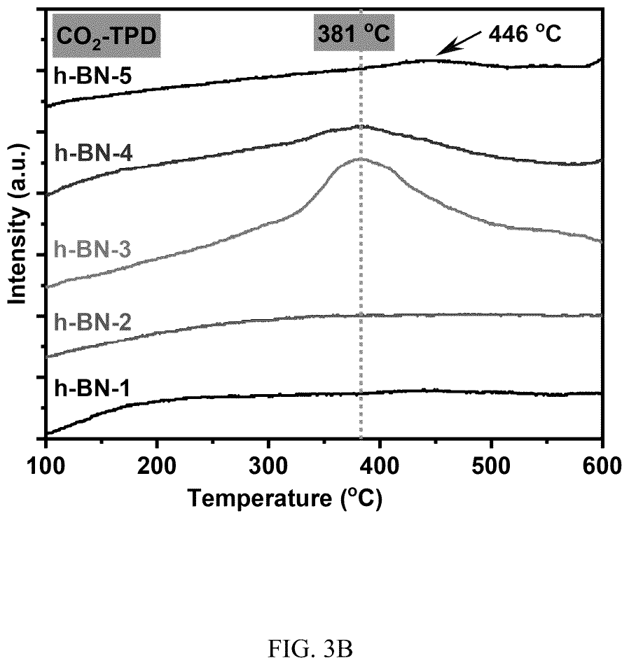

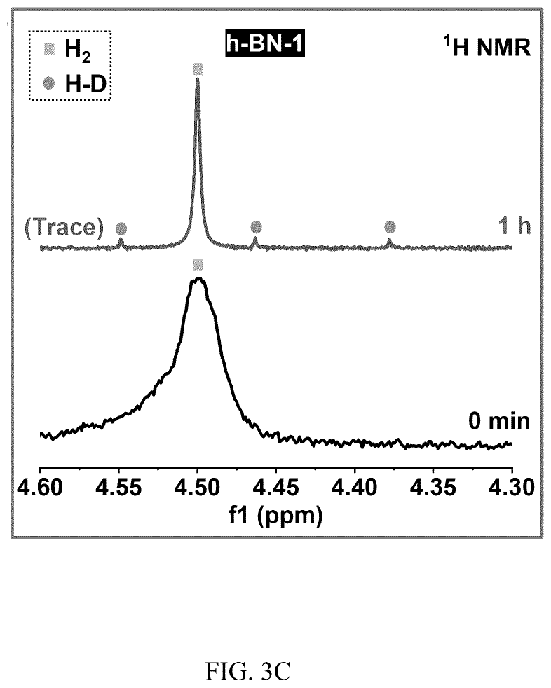

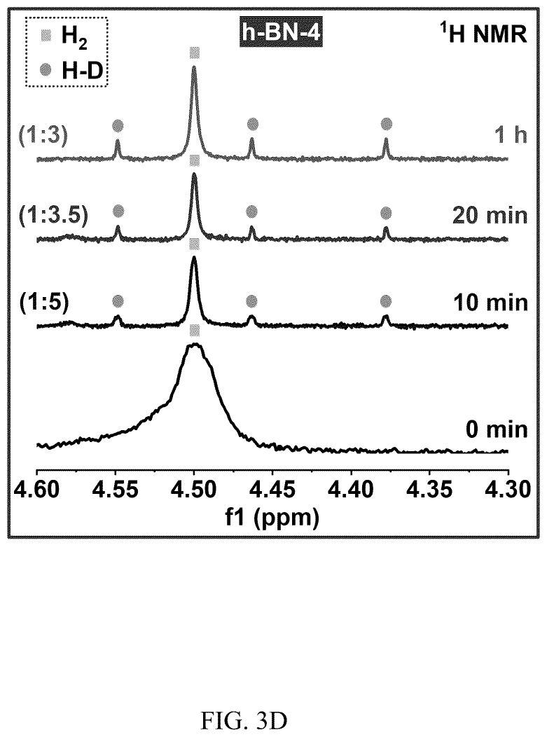

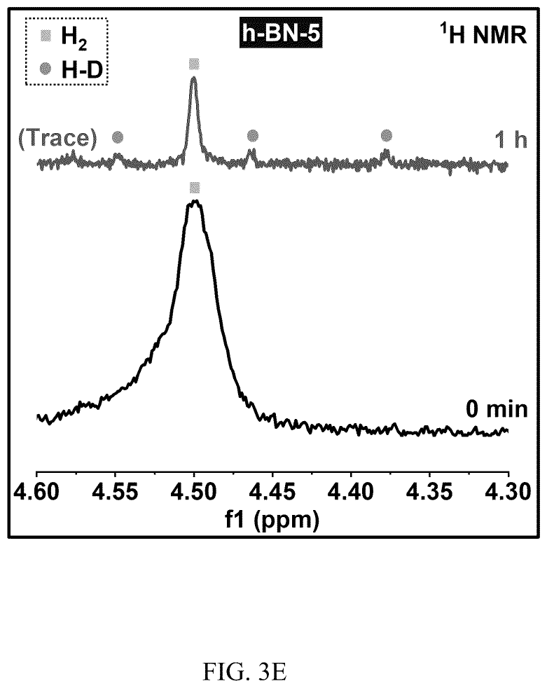

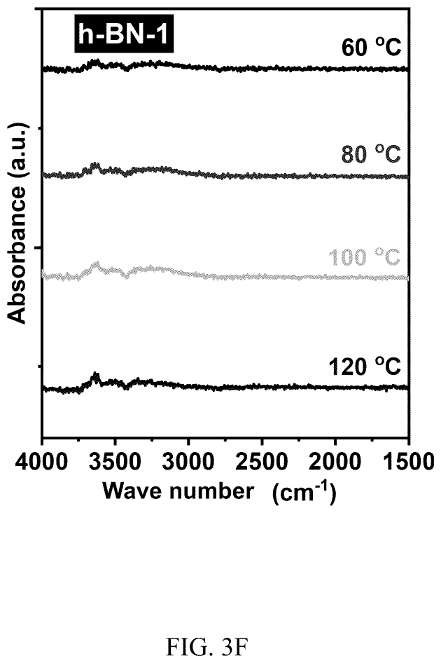

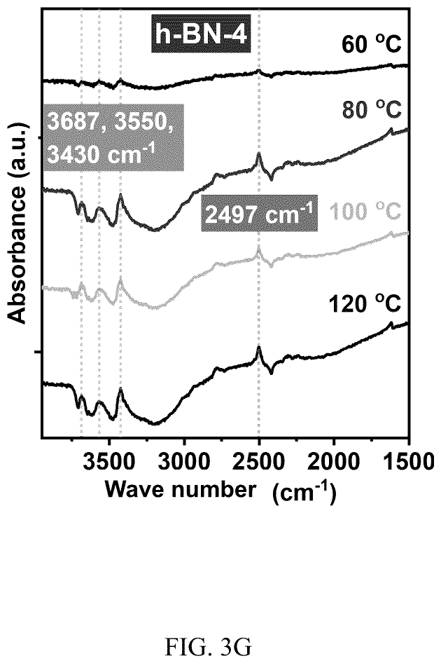

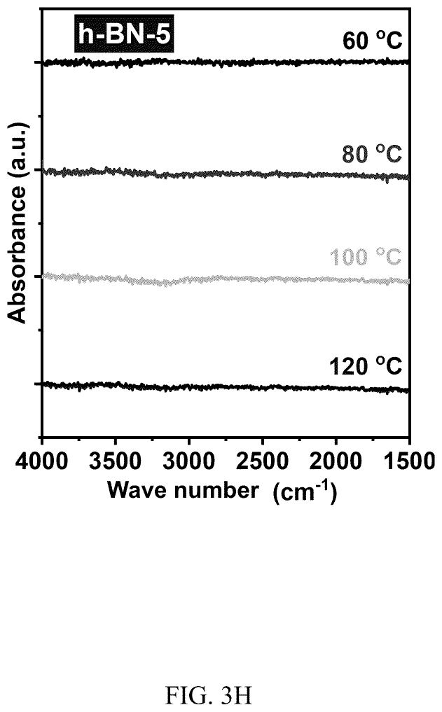

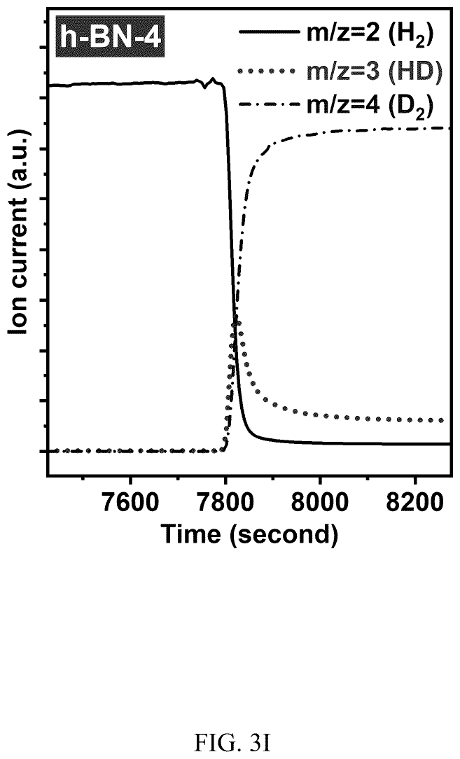

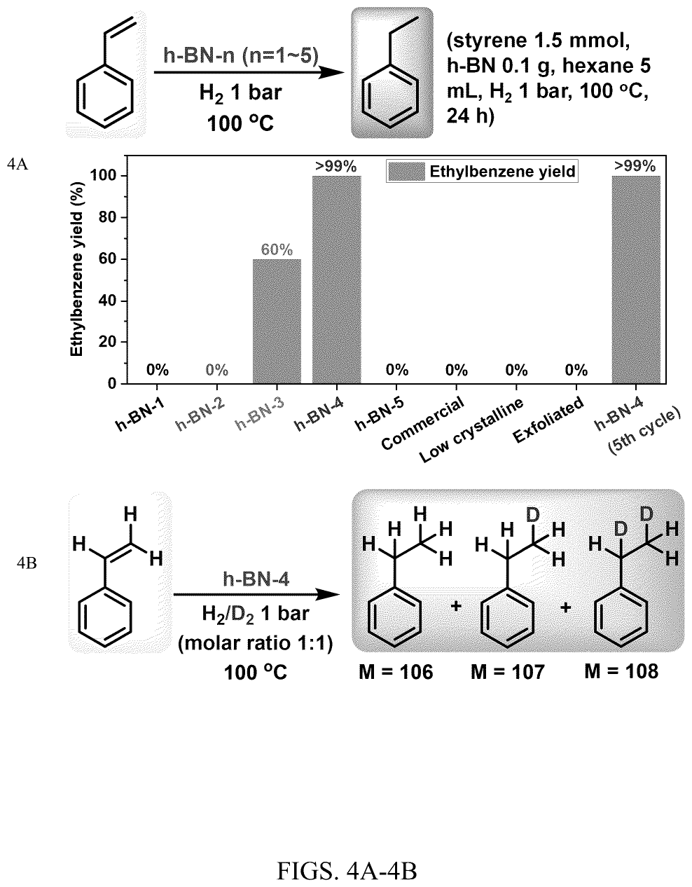

To evaluate the H₂ activation potential of these h-BN materials, their surface basicity and acidity properties were measured by temperature programmed desorption (TPD) profiles. As shown in FIG. 3A, no signals were present in the NH₃-TPD profiles of h-BN-1 and h-BN-2, which indicates no active Lewis acid sites in their skeletons. Comparatively, in h-BN-3, a small peak assigned to the unsaturated B centers (Lewis acid sites) appeared at 385° C., and the intensity of the corresponding peak significantly increased in the NH₃-TPD profile of h-BN-4, which demonstrates a high content of unsaturated B center in h-BN-4. No NH₃ adsorption was achieved by h-BN-5. As revealed by the CO₂-TPD profile in FIG. 3B, h-BN-1 and h-BN-2 contained no active Lewis basic sites for CO₂ adsorption. A characteristic peak located around 381° C. in the CO₂-TPD profile of h-BN-3 was afforded by the unsaturated N sites (Lewis base), which was also present in h-BN-4 but at slightly lower content. A small amount of Lewis basic N sites were created in h-BN-5 with relatively stronger basicity (CO₂-TPD: 446° C.). Therefore, the coexistence of Lewis acidic and basic sites was observed in the skeleton of h-BN-3 and h-BN-4, which may display H₂ activation capability, and this has been revealed by their catalytic activity in styrene hydrogenation, as shown in FIG. 4A.

To demonstrate the H₂ activation capability of h-BNs and demonstrate their FLP behavior, H/D isotope scrambling experiments were performed using an H₂/D₂ mixture (ca. 1:1 v/v) (1 bar) at 100° C. taking h-BN-4 as an example. The results are shown in FIGS. 3C-3E. FIG. 3C shows H/D exchange detected by ¹H NMR (d₁₂-cyclohexane, 400 MHZ) at 100° C. and 1 bar H₂/D₂ (1:1) catalyzed by h-BN-1. FIG. 3D shows H/D exchange detected by ¹H NMR (d₁₂-cyclohexane, 400 MHZ) at 100° C. and 1 bar H₂/D₂ (1:1) catalyzed by h-BN-4. FIG. 3E shows H/D exchange detected by ¹H NMR (d₁₂-cyclohexane, 400 MHZ) at 100° C. and 1 bar H₂/D₂ (1:1) catalyzed by h-BN-5. When h-BN-4 was dispersed in d₁₂-cyclohexane, after bubbling with H₂/D₂, only the signal for H₂ (8=4.54 ppm) was observed in the ¹H NMR spectra. A thermal treatment of the mixture for only 10 min at 100° C. led to the formation of HD, as demonstrated by the emerging triplet centered at 4.50 ppm, with the H₂:HD molar ratio of 1:5 (FIG. 3D). Further increasing the reaction time to 20 minutes resulted in more HD formation with an H₂:HD molar ratio of 1:3.5, which was slightly increased to 1:3 by prolonging the reaction time to 1 hour. Comparatively, only a trace amount of HD was detected in the presence of h-BN-1 (FIG. 3C) and h-BN-5 (FIG. 3E) within 1 hour, which demonstrates the critical role of varying the feeding ratio of NaBH₄:NaNH₂ for controlling the defects in h-BN. The better performance of h-BN-4 can be attributed to the relatively high content of fixed unsaturated B and N centers as reflected by the TPD results (see, e.g., FIGS. 3A and 3B).

In situ diffuse reflectance infrared Fourier transform spectroscopy (DRIFTS) of H₂ chemisorption was conducted to study the H-H cleavage capability of h-BN skeletons in h-BN-1, h-BN-4, and h-BN-5. The results are shown in FIGS. 3F, 3G, and 3H, which show in situ DRIFTS spectra of H₂ chemisorption on h-BN-1, h-BN-4, and h-BN-5, respectively. After pretreatment of the h-BNs in flowing 10% N₂/Ar at 400° C. and cooling down to room temperature, pure H₂ flow was passed through in the temperature range of 60-120° C. For h-BN-1, only some weak N—H stretching mode (3300-3600 cm⁻¹) appeared (see FIG. 3F).

Comparatively, for h-BN-4, as the temperature increased, three peaks located at 3430, 3550, and 3687 cm⁻¹ were observed, being assigned to the N—H stretching mode (see FIG. 3G). The characteristic B-H stretching mode at 2497 cm⁻¹ was significant, indicating successful cleavage of H-H by N and B moieties within the skeleton of h-BN-4. Consistent with the H/D isotope scrambling experiments detected by liquid NMR, h-BN-5 exhibited no significant peaks during H₂ treatment (see FIG. 3H). The H/D isotope scrambling experiment, which was employed to demonstrate FLP behavior in a material, further confirmed the superior H₂ activation capability of h-BN-4. FIG. 3I is a graph showing mass fragments of the outlet gas stream derived from H₂/D₂ chemisorption on h-BN-4. As shown in FIG. 3I, the superior H₂ activation capability of h-BN-4 is demonstrated by the presence of HD (m/z=3) in the outlet gas stream upon changing the feeding gas from H₂ to D₂ at 120° C.

Hydrogenation of styrene was selected as a feature reaction to explore the catalytic performance of defect-containing h-BN. FIG. 4A provides a schematic and data showing catalytic activity of h-BNs in styrene hydrogenation. FIG. 4B is a schematic showing isotope-labeling experiments catalyzed by h-BN-4 under H₂/D₂ atmosphere. As shown in FIG. 4A, when h-BN-1 or h-BN-2 was deployed, no styrene conversion was detected under 1 bar H₂ at 100° C. within 24 h. In comparison, h-BN-3 afforded a 60% yield of ethylbenzene, which demonstrates that H₂ activation can be achieved by tuning the vacancy properties of h-BN materials. Notably, as further shown in FIG. 4A, the catalytic performance was further improved by h-BN-4, with >99% yield of ethylbenzene being achieved under ambient H₂ pressure. However, as also shown in FIG. 4A, h-BN-5 yielded no hydrogenation activity. Notably, the catalytic performance of h-BN-4 was in accordance with the H₂ activation results via H/D exchange and DRIFTS spectra of H₂ chemisorption.

The catalytic activity of h-BN materials fabricated by other methods was also tested for styrene hydrogenation ability. Commercial h-BN showed no catalytic activity. The h-BN counterpart prepared from boron trioxide and urea with relatively low crystallinity possessed no ability to activate H₂ under ambient pressure (H. Chen et al., Adv. Funct. Mater., 29 (50), 1906284, 2019). The defects-enriched h-BN nanosheets (h-BNNS) derived from liquid N₂ exfoliation gave no yield of ethylbenzene, underscoring the importance of carefully selecting the defect types by successively varying the feeding ratio of B and N source. TPD profiles of these non-active h-BNs revealed the lack of Lewis acid or base sites within their scaffolds.

Notably, the excellent H₂ activation performance of h-BN-4 derived herein was further demonstrated by its good reusability, with >99% yield of ethylbenzene being maintained after cycling for five times. An isotope labeling experiment with an H₂/D₂ (molar ratio of 1:1) mixture in styrene hydrogenation afforded ethylbenzene mixtures with molecular weights (MWs) of 106, 107, and 108, corresponding to the involvement of none, one, and two D atoms in the product, respectively. Successful cleavage of the H-H and D-D bond was verified by the ethylbenzene product with MW=107 derived from the addition of H and D atom (see FIG. 4B). Theoretical calculations based on density functional theory (DFT) were conducted and the results revealed that a h-BN model with a medium sized BN2 vacancy was capable of efficiently activating H₂ via a FLP-like behavior and achieving the subsequent styrene hydrogenation with moderate activation energies.

In summary, the creation of sterically hindered Lewis acid and base sites in h-BN skeleton was achieved by controlling the feeding ratio of boron and nitrogen sources via the molten salts-derived ionothermal method. The as-afforded h-BNs were able to achieve efficient H₂ activation and dissociation under atmosphere pressure via FLP-like behavior. Attractive performance of the corresponding h-BN in a hydrogenation reaction further elucidated that promising progress has been made in this work to construct high-quality metal-free heterogeneous catalysts towards industrial hydrogenation procedures.

While there have been shown and described what are at present considered the preferred embodiments of the invention, those skilled in the art may make various changes and modifications which remain within the scope of the invention defined by the appended claims.

What is claimed is:

1. A composition comprising a boron nitride hexagonal lattice structure in which boron atoms and nitrogen atoms are present in a B:N molar ratio of 1:4-1:8 or 4:1-8:1, wherein said molar ratio corresponds to vacant site defects within the boron nitride hexagonal lattice structure.

2. The composition of claim 1, wherein the B:N molar ratio is 1:5-1:7 or 5:1-7:1.

3. The composition of claim 1, wherein the B:N molar ratio is 1:4-1:8.

4. The composition of claim 1, wherein the B:N molar ratio is 1:5:1:7.

5. The composition of claim 1, wherein the composition is prepared by a method in which a molten mixture of NaBH₄ and NaNH₂ is heated under an inert atmosphere to a temperature of at least 700° C., wherein the molar ratio of NaBH₄:NaNH₂ is 1:4-1:8 or 4:1-8:1.

6. The composition of claim 5, wherein the temperature is at least 800° C.

7. The composition of claim 5, wherein the molar ratio of NaBH₄:NaNH₂ is 1:5:1:7 or 5:1-7:1.

8. The composition of claim 5, wherein the molar ratio of NaBH₄:NaNH₂ is 1:4-1:8.

9. The composition of claim 5, wherein the molar ratio of NaBH₄:NaNH₂ is 1:5:1:7.

10. A method of producing a boron nitride hexagonal lattice structure containing vacant site defects, the method comprising heating a molten mixture of NaBH₄ and NaNH₂ under an inert atmosphere to a temperature of at least 700° C., wherein the molar ratio of NaBH₄:NaNH₂ is 1:4-1:8 or 4:1-8:1.

11. The method of claim 10, wherein the temperature is at least 800° C.

12. The method of claim 10, wherein the molar ratio of NaBH₄:NaNH₂ is 1:5:1:7 or 5:1-7:1.

13. The method of claim 10, wherein the molar ratio of NaBH₄:NaNH₂ is 1:4-1:8.

14. The method of claim 10, wherein the molar ratio of NaBH₄:NaNH₂ is 1:5:1:7.

15. A method of catalytically hydrogenating an unsaturated organic compound, the method comprising contacting the unsaturated organic compound with hydrogen gas in the presence of a hydrogenation catalyst at a temperature of 80° C.-300° C., wherein the hydrogenation catalyst comprises a boron nitride hexagonal lattice structure in which boron atoms and nitrogen atoms are present in a B:N molar ratio of 1:4-1:8 or 4:1-8:1, wherein said molar ratio corresponds to vacant site defects within the boron nitride hexagonal lattice structure.

16. The method of claim 15, wherein said temperature is 80° C.-200° C.

17. The method of claim 15, wherein said temperature is 80° C.-150° C.

18. The method of claim 15, wherein the B:N molar ratio is 1:5:1:7 or 5:1-7:1.

19. The method of claim 15, wherein the B:N molar ratio is 1:4-1:8.

20. The method of claim 15, wherein the B:N molar ratio is 1:5:1:7.
